(19) United States
(12) Patent Application Publication
(10) Pub. No.: US 2023/0383262 A1
(43) Pub. Date: Nov. 30, 2023

(54) METHOD FOR CREATING CELL SPHEROIDS

(71) Applicant: THE ROYAL INSTITUTION FOR THE ADVANCEMENT OF LEARNING / MCGILL UNIVERSITY, Montrèal (CA)

(72) Inventors: Mohammadreza RASOULI, Montreal (CA); Maryam TABRIZIAN, Longueuil (CA)

(21) Appl. No.: 18/198,953

(22) Filed: May 18, 2023

Related U.S. Application Data
(60) Provisional application No. 63/343,814, filed on May 19, 2022.

(51) Int. Cl. C12N 5/09; C12N 13/00; C12M 3/06; C12M 1/42

(52) U.S. Cl. CPC C12N 5/0693; C12N 13/00; C12M 23/16; C12M 35/04; C12N 2533/54; C12N 2513/00

(57) ABSTRACT

A method for forming cell spheroids in a fluidic system, includes: injecting a mixture including cells embedded in a biomaterial matrix into a channel; generating vortices in the mixture flowing within the channel; trapping the cells using the vortices to form clusters of cells until the cells of the clusters of cells adhere to one another via the biomaterial matrix thereby forming the cell spheroids; and retrieving the cell spheroids from the channel.

### CROSS-REFERENCE TO RELATED APPLICATIONS

This application claims priority from U.S. patent application 63/343,814 filed on May 19, 2022, the entire contents of which are incorporated herein by reference.

### TECHNICAL FIELD

This disclosure relates generally to small-scale fluidic systems, such as microfluidic systems or millifluidic systems and, more particularly, to systems and methods used for generating cell spheroids in such fluidic systems.

### BACKGROUND

Multicellular spheroids are believed to be excellent models to replicate the physiological functions, structural complexities of living tissues, and their native configuration. Their 3D architecture and obviation of cell-substrate interaction in such environment may allow for faithful recapitulation of biochemical and biomechanical communication between cell-cell and cell-matrix. These unique characteristics may render spheroids the optimal candidate for numerous fundamental studies and biomedical applications, including the development of pre-clinical models for drug discovery, regenerative medicine, and tissue engineering.

Spheroids of cancer cells, also known as tumoroids, may be used for the investigation of anticancer therapeutics' response, as they provide analogous spatial architecture, diffusion gradient, tumor dynamics, metabolic activity, and drug resistance behavior of solid tumors. In a similar vein, spheroids of stem cells may offer higher cell viability, proliferation, stemness, and regenerative characteristic compared to 2D culture. Cell spheroids may be used as tissue engineering building blocks to replace single-cell printing, where their complex composition, prolonged survival and fusion capacity are used to reconstruct various tissues, from branched blood vessel to thyroid gland and osteochondral interface.

The scalable application of spheroids in the above-mentioned studies necessitates a high-throughput production method with consistent physiological and morphological characteristics. Existing methods are generally labor-intensive, low-yield, time-consuming, and show heterogeneous spheroids in shape and size due to poor control of the process which limits their scaled-up application.

Microfluidics has shown the capacity to overcome some of the technical hurdles in spheroid formation by offering controlled physical conditions, minimized cells and reagent consumption, high sensitivity in drug screening, precise manipulation of cells, continuous perfusion, and regulation of the nutrients and oxygen supply.

The main mechanism of spheroid generation in the majority of microfluidic platforms is based on the physical arrangement of cells and promoting direct cell-cell contact by applying different forces. Existing methods rely on the gradual secretion of adhesive proteins by cells to develop clusters into spheroids. Thus, these methods usually take hours if not days, depending on cell types. Moreover, during this long incubation time, cells can develop adhesion to the channel walls which makes the spheroids' retrieval challenging.

### SUMMARY

In one aspect, there is provided a method for forming cell spheroids in a fluidic system, comprising: injecting a mixture including cells embedded in a biomaterial matrix into a channel; generating vortices in the mixture flowing within the channel; trapping the cells using the vortices to form clusters of cells until the cells of the clusters of cells adhere to one another via the biomaterial matrix thereby forming the cell spheroids; and retrieving the cell spheroids from the channel.

The method as defined above and described herein may also include any one or more of the following features, in whole or in part, and in any combination.

In some embodiments, the generating of the vortices includes generating acoustic vibrations in the mixture to form acoustic microstreams in the mixture.

In some embodiments, the generating of the vortices using the acoustic vibrations includes inducing vibrations of sharp edges extending within the channel with a piezo transducer.

In some embodiments, the trapping of the cells into the clusters of cells includes continuously flowing the mixture within the channel while the cell spheroids are being formed.

In some embodiments, the generating of the vortices includes generating the vortices with acoustic vibrations.

In some embodiments, the method comprises stopping the acoustic vibrations once the cell spheroids reach a desired size, thereby allowing the cell spheroids to move toward an outlet of the channel.

In some embodiments, the injecting of the mixture includes injecting the mixture including the cells embedded in a collagen mixture.

In some embodiments, the collagen mixture includes Type I collagen.

In some embodiments, the method comprises preparing the collagen mixture by: obtaining a solution of acid solubilized collagen; and neutralizing the solution with sodium hydroxide to obtain the collagen mixture.

In some embodiments, the injecting of the mixture includes injecting the mixture having a cell concentration of from 0.3 to 2 million cells per millilitre.

In some embodiments, the retrieving the cell spheroids includes flowing the cell spheroids out of the channel by injecting a fluid into the channel to push the cell spheroids towards an outlet of the channel.

In some embodiments, the injecting of the fluid includes injecting phosphate-buffered saline or Dulbecco's modified Eagle medium.

In some embodiments, the method comprises introducing methylcellulose into the mixture.

In some embodiments, the methylcellulose is introduced into the mixture prior to the injection of the mixture into the channel.

In some embodiments, the methylcellulose is introduced into the mixture after the mixture has been injected into the channel.

In another aspect, there is provided a method for forming cell spheroids in a fluidic system, comprising: using acoustic vibrations to generate vortices in a fluid mixture within the fluidic system, the fluid mixture including cells embedded in a biomaterial matrix; and trapping clusters of cells using the vortices, the clusters of cells adhering to one another thereby forming the cell spheroids.

The method as defined above and described herein may also include any one or more of the following features, in whole or in part, and in any combination.

In some embodiments, the method comprises forming acoustic microstreams in the mixture using the vortices.

In some embodiments, the method comprises using a piezo transducer to induce vibrations of sharp edges within a channel containing the fluid mixture.

In some embodiments, the method comprises continuously flowing the fluid mixture through the channel while the cell spheroids are being formed.

In some embodiments, the method comprises injecting a methylcellulose into the fluid mixture.

In one aspect, there is provided a method for generating cell spheroids, comprising: injecting a mixture including cells embedded in a biomaterial matrix into a channel; generating vortices in the mixture flowing within the channel; trapping the cells using the vortices to form clusters of cells until the cells of the clusters of cells are adhered to one another via the biomaterial matrix thereby forming the cell spheroids; and flowing the cell spheroids out of the channel to retrieve the cell spheroids.

The method as defined above and described herein may also include any one or more of the following features, in whole or in part, and in any combination.

In some embodiments, the injecting of the mixture includes injecting the mixture including the cells embedded in a collagen mixture, True gel 3D, gel MA, alginate, poly-L-lysine, or Type I collagen.

In some embodiments, the biomaterial is Type I collagen, the method comprising preparing the collagen mixture by: obtaining a solution of acid solubilized collagen; and neutralizing the solution with sodium hydroxide to obtain the collagen mixture.

In some embodiments, the trapping of the cells into the clusters of cells includes continuously flowing the mixture within the channel while the cell spheroids are being formed.

In some embodiments, the generating of the vortices includes generating the vortices with acoustic vibrations, the method comprising stopping the acoustic vibrations once the cell spheroids reach a desired size thereby allowing the cell spheroids to move toward an outlet of the channel.

In some embodiments, the injecting of the mixture includes injecting the mixture having a cell concentration of from 0.3 to 2 million cells by millilitre.

In some embodiments, the generating of the vortices includes generating the vortices using acoustic vibrations.

In some embodiments, the generating of the vortices using the acoustic vibrations includes inducing vibrations of sharp edges extending within the channel with a piezo transducer.

In some embodiments, the flowing of the cell spheroids out of the channel includes injecting a fluid into the channel to push the cell spheroids towards an outlet of the channel.

In some embodiments, the injecting of the fluid includes injecting phosphate-buffered saline or Dulbecco's modified Eagle medium.

In some embodiments, the method includes introducing methylcellulose into the mixture.

### BRIEF DESCRIPTION OF THE DRAWINGS

Reference is now made to the accompanying figures in which:

FIG. 1 is a schematic three dimensional view of an acoustic mixer in accordance with a particular embodiment;

FIG. 2 is an enlarged view of a portion of FIG. 1;

FIG. 3 is a front view of an enlarged portion of FIG. 2;

FIG. 4 is a flowchart illustrating steps of a method for creating cell spheroids;

FIG. 5 is a flowchart illustrating steps of a method for forming cell spheroids;

FIG. 6 is a graph illustrating a relation between a flow rate through the acoustic mixer of FIG. 1 and a voltage supplied to a piezo transducer of the mixer of FIG. 1;

FIG. 7 illustrates the formation of cell spheroids at a plurality of time stamps; and

FIG. 8 is a three dimensional view of a portion of the acoustic mixer of FIG. 1 illustrating schematically the formation of the cell spheroids.

### DETAILED DESCRIPTION

In the present disclosure, a rapid spheroid formation method is disclosed and is based on boundary-driven acoustic streaming to produce compact cell-collagen aggregates. Acoustically-induced hydrodynamic forces may agglomerate cells into compact clusters in a span of seconds and allow real-time monitoring and controlling the size of the cell aggregates. As will be described below, the propagation of the acoustic wave(s) in the platform is converted into a strong set of counter-rotating microstreams. The hydrodynamic forces that stem from these microstreams may trap cells and form cell clusters, as the initial step of spheroid formation. Since the cell clusters are initially loose, strong acoustic microstreams are simultaneously used to incorporate a matrix for rapid coagulation of cells, and hence, accelerate the stage of matrix development to seconds.

Primarily, spheroid formation includes a cultivation system for the physical aggregation of the cells. The proposed platform employs boundary-driven acoustic streaming to convene cells into the compact vicinity of each other to form clusters. These acoustic vortices are the result of acoustic energy dissipation in a thin boundary layer around oscillatory solid-liquid or gas-liquid interfaces.

In this disclosure, an acoustic mixer that may be used to generate cell spheroids is described first followed by a method for generating the cell spheroids that may use the acoustic mixer.

### Acoustic Mixer

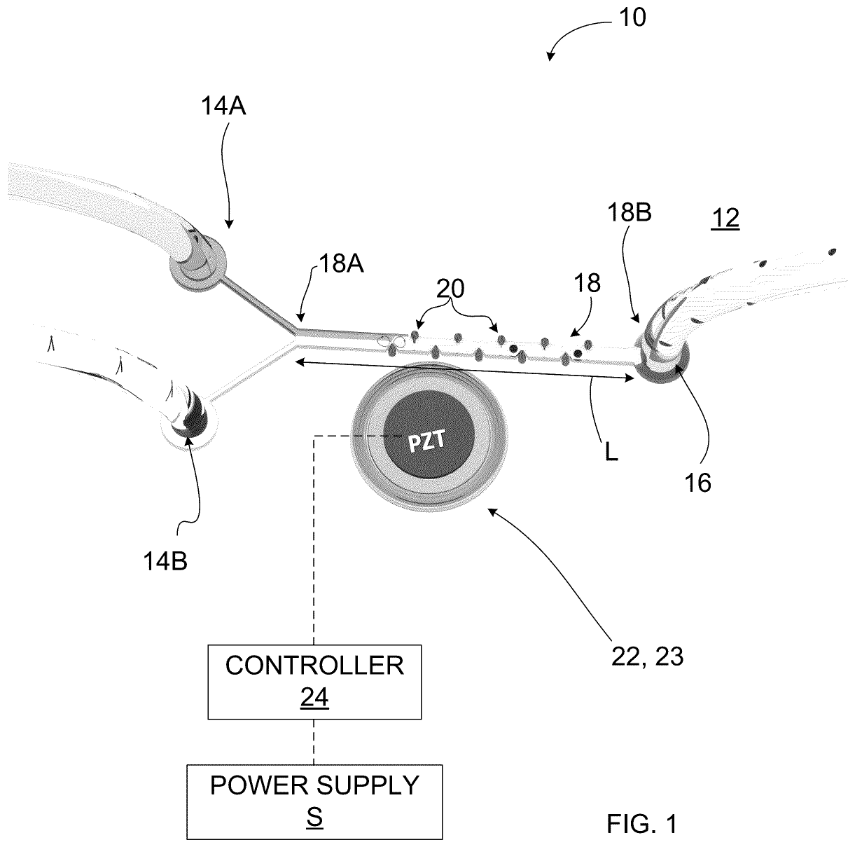

Referring now to FIGS. 1-3, an acoustic mixer is generally shown at 10. The acoustic mixer 10 is configured to incorporate two mechanisms (i.e., bubble and sharp edges). In the depicted embodiment, the acoustic mixer 10 includes a chip 12. Two inlets 14A, 14B an and an outlet 16 defined through the chip 12. Each of the two inlets 14A, 14B is configured for receiving a respective one of two fluids to be mixed. The outlet 16 is configured for outputting a mix of the two fluids. In some embodiments, only one inlet may be used. The mixer 10 includes a mixing channel, also referred to as a microfluidic channel, 18 defined by the chip 12. The channel 18 may also be a millifluidic channel. The channel 18 may, in one particular embodiment, have a width of about 600 micrometers, but may alternatively have a width up to a few millimetres. The mixing channel 18 extends from a channel inlet 18A fluidly connected to the two inlets 14A, 14B for receiving the two fluids to be mixed and a channel outlet 18B spaced apart from the channel inlet 18A, fluidly connected to the outlet 16. The channel as defined herein may therefore be within the micro or mille scale.

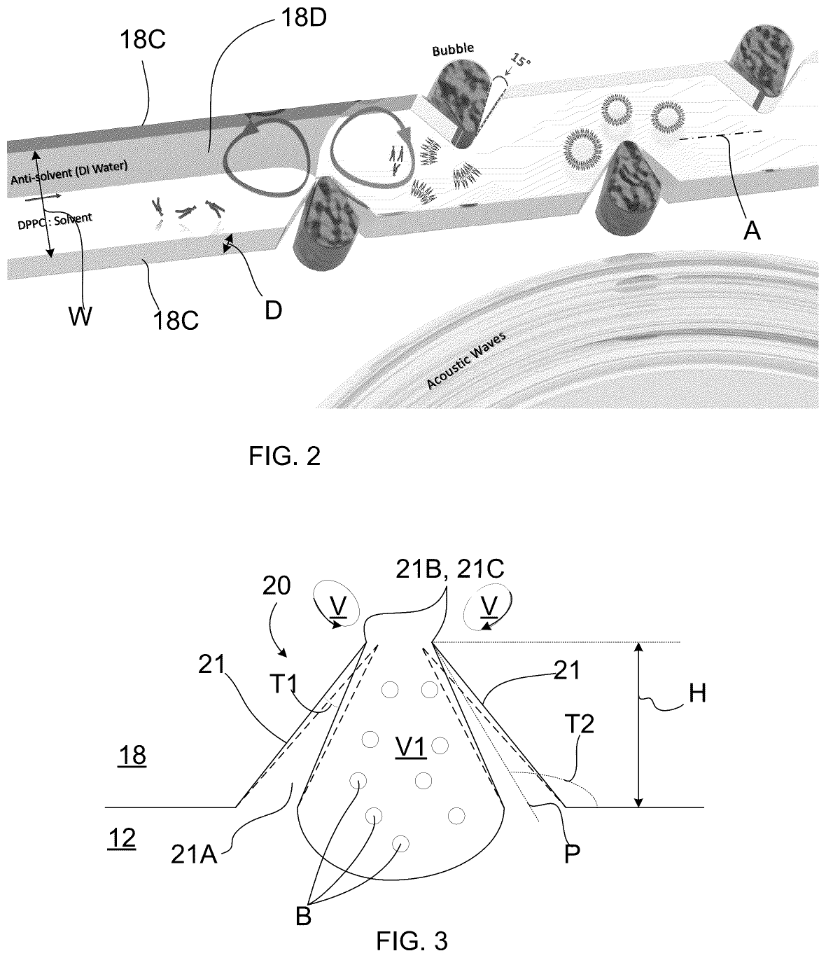

Referring more particularly to FIG. 2, in the depicted embodiment, the mixing channel 18 includes side walls 18C and top and bottom walls 18D extending from one of the two side walls 18C to the other. In the depicted embodiment, a cross-section of the mixing channel 18 taken on a plane normal to a longitudinal axis A has a rectangular shape. Other shapes are contemplated. The mixing channel has a length L of about 1.2 cm in the depicted embodiment, a width W of about 600 μm in the depicted embodiment, and a depth D in a direction transverse to the width W and to the length L of about 100 μm, preferably 250 μm, in the depicted embodiment. The width W of the mixing channel 18 is defined from one of the two side walls 18c to the other whereas the depth D is defined from the top wall to the bottom wall 18d. In the embodiment shown, a width W over depth D (W/D) ratio is about 6, a length L over width W (L/W) ratio is about 20, and a length L over depth D (L/D) ratio is about 120. Other dimensions are considered without departing from the scope of the present disclosure.

The flow channel 18 is optionally manufactured using polydimethylsiloxane, but other materials such as polybutylene terephthalate (PBT), polycarbonates, polystyrene, Glass, Quartz, lithium niobate, polypropylene, elastomeric polymers, steel, and thermoplastics are contemplated.

Referring to FIG. 3, the acoustic mixer 10 includes at least one pair of mixing inducing features 20, ten in the embodiment shown, five on each of the side walls 18C, which, in the embodiment shown, are pairs of protrusions 21 extending from each the two side walls 18c of the mixing channel 18 toward the other. In the embodiment shown, the pairs of mixing inducing features 20 are axially offset from one another relative to the longitudinal axis A and disposed in alternation on the two side walls 18C of the mixing channel 18 along the longitudinal axis A. In other words, the structures (e.g., pairs of mixing inducing features 20) are positioned on the two side walls 18C asymmetrically so that acoustic vortices traverse fluids interface and transport mass between two fields.

In the embodiment shown, the protrusions 21 are cantilevered and extend from roots 21A secured to one of the two side walls 18C to tips 21B located between the two side walls 18C. A cross-section of each of the protrusions 21 taken on a plane intersecting both of the two side walls 18C is triangular. Stated differently, the protrusions 21 tapers from their roots 21A to their tips 21B to define sharp edges 21C at their tips 20B. In the embodiment shown, each of the protrusions 21 has a height H extending from their roots 21A to their tips 21B that may be about 250 μm, preferably 300 μm in the present embodiment. In the embodiment shown, a height H over width W (H/W) ratio is about 0.42.

In the embodiment shown, a tip angle T1 of the protrusions 21, which is defined between two walls of the protrusions 21 that meet at the tips 21B of the sharp edges 21C, is about 15 degrees and is chosen as to be optimum for microstreaming and is within current fabrication limits. The tip angle T1 may range from greater than 0 to 80 degrees, and may be from 15 to 80 degrees in some embodiments. In the depicted embodiment, a distance along the longitudinal axis A between the two protrusions 21 of each pairs of mixing inducing features 20 decreases from their roots 21A to their tips 21B. In the embodiment shown, a slanting angle T2 of the protrusions 21 ranges from 30 to 90 degrees. The slanting angle T2 extends from the side wall 18C to a mid-plane P of the protrusions 21.

In the depicted embodiment, the protrusions 21 are slanted so that there will be a sequestered volume V1 between the protrusions 21 of each of the pairs of protrusions. In this sequestered volume V1, air bubbles can be confined upon passage of fluids, due to low surface tension with hydrophobic channel sides. For simplification, the combination of two sharp-edges 21C and the bubble B contained within their respective volume V1 between the two protrusions 21 is referred to as the combined unit. The combined units are positioned on the upper side and lower side asymmetrically so that acoustic vortices traverse fluids interface and transport mass between two fields.

Referring back to FIG. 1, the acoustic mixer 10 further includes a vibration generating device 22 secured to the chip 12 for inducing vibrations of the mixing inducing features 20. In the embodiment shown, the vibration generating device 22 is a piezo transducer 23, but any other suitable vibration generating device 22, such as acoustic vibrator and so on may be used. The piezo transducer 23 is operatively connected to a controller 24 and to a power supply S for generating the vibrations and for controlling the frequency and/or amplitudes of said vibrations. In the embodiment shown, the controller 24 is used for generating harmonic electrical signals that may be initiated by a function generator (e.g., AFG3011C, Tektronix, USA) which also governs the signal's frequency and waveform. The function generator may then be connected to an amplifier (e.g., Amplifier Research, USA) to regulate the amplitude of the voltage and transmit the signal to the piezo transducer 23. In the embodiment shown, the electrical impedance of the piezo transducer 23 was evaluated with Agilent 4294A impedance analyzer (Agilent, Palo Alto, CA). The spectra of 40 Hz-100 kHz was explored using 201 nodes. The piezo-elements were mounted on the chip and connected to low and high voltage terminals with the peak to peak amplitude of 1 Vpp. The controller 24 may include a processing unit and a computer-readable medium operatively connected to the processing unit and comprising instructions executable by the processing unit. The frequency of the vibrations may be about 16.1 kHz. Herein, the expression “about” implies variations of plus or minus 10%.

The piezo transducer 23 may polarize in Z-direction in a synchronized response to the electric excitation; however, the oscillation occurs in all directions owing to both direct and transverse effects. Via this oscillation, the electric energy transforms to acoustic energy. The acoustic energy then propagates through acoustic waves in the glass substrate of the chip 12 and the PDMS layer and manifests as high-amplitude mechanical vibration in sharp edges 20C and bubble membrane. Finally, the vibration induces the boundary layer microstreaming phenomenon described in detail in the geometry section. The protrusions 21 are shown in FIG. 3 in two different positions (solid and dashed lines) illustrating an amplitude of their vibratory motions.

The acoustic mixer 10 may be manufactured through photolithography, followed by single-layer soft lithography. Any suitable process known in the art may however be used to manufacture the acoustic mixer 10 without departing from the scope of the present disclosure. In the embodiment shown, a negative photoresist (SU-8 2050 Micro Chem Corp., USA) was spin-coated on a silicon wafer, per the manufacturer protocol, to fabricate the master of 100-micron thickness. Polydimethylsiloxane (PDMS) was poured on the silanized master to replicate the pattern of the channels and microstructures by soft lithography. The patterned PDMS was bonded on a glass substrate by plasma surface treatment. A piezoelectric transducer 23 (model no. 273-073, Radioshack) was then mounted on the glass substrate along the side of the PDMS microchannels to complete the assembly of the microfluidic chip. In the embodiment shown, the devices were then treated with a step of Parylene C coating. In the embodiment shown, the deposition was conducted in SCS Labcoter 2 PDS 2010 (Specialty Coating Systems, USA) with 2 grams of Parylene-C dimers which corresponds to a coating thickness of 1 μm.

In some embodiments, the acoustic mixer 10 may include a plurality of channels 18. This may allow the generation of cell spheroids in parallel within each of the channels. Only one, or more than one piezo transducer may be used to generate the vibrations that create the vortices V.

### Method

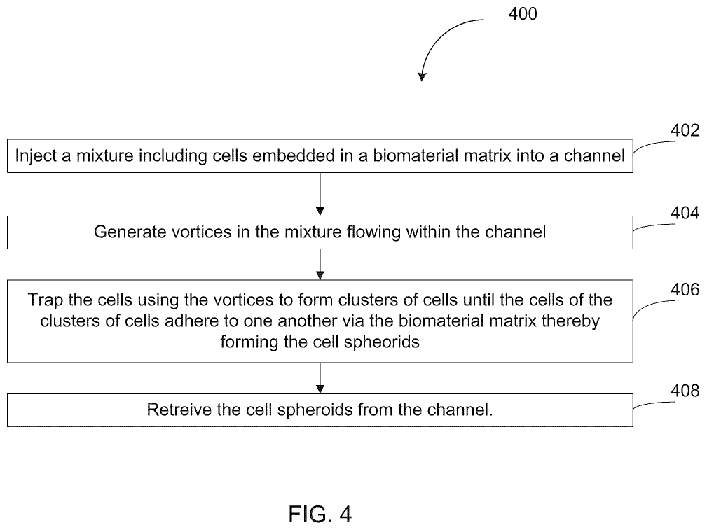

Referring now to FIG. 4, a method of forming cell spheroids in a fluidic system is shown at 400. The method 400 includes injecting a mixture including cells embedded in a biomaterial matrix into the mixing channel 18 at 402; generating vortices in the mixture flowing within the mixing channel 18 at 404; trapping the cells using the vortices to form clusters of cells until the cells of the clusters of cells adhere to one another via the biomaterial matrix thereby forming the cell spheroids at 406; and retrieving the cell spheroids from the channel 18 at 408.

### Mixture

In the embodiment shown, injecting of the mixture including the biomaterial matrix, at 402, includes injecting the mixture including the cells embedded in the biomaterial matrix including one or more of a collagen mixture, True gel 3D, gelMA, alginate, poly-L-lysine, or Type I collagen.

To develop sturdy cell-cell binding and transform them into spheroids, the cell clusters need to tether together through the secretion of adhesion protein molecules. Otherwise, the cells will disperse and follow the flow trajectory as soon as the acoustic source stops. Relying on cells to secret the ECM to stabilize the cell-cell adhesion for the formation of spheroids, can take hours and it varies among cell types.

To circumvent this delay phase, a bioadhesive matrix is needed to glue cells as they convene in the acoustic trap. In searching for the compatible media for acoustic assembly, we explored a few biocompatible and frequently used bioadhesives to assess their performances with respect to: i) instantaneous adhesion and robustness for holding cells together; ii) easy spheroids' retrieval by pre-venting spheroids' adhesion to the PDMS channel sidewalls; and iii) formability of cell aggregates into spheroids under acoustic microstreams.

First, the spheroid formation was tested under stop-flow conditions for over an hour to provide cells with some time to form clusters. Second, the acoustic microstreams were switched off after the initial cell trapping because acoustic streams created cells' mobility that would not allow them to attach together. Third, since cells may gradually dispersing in stagnant conditions, methylcellulose (MC) is added in the stop-flow condition. Its high viscosity allowed to confine cells and helped them to attach to each other in 30 min to 1 h. Results showed that the use of MC played a role for parallel spheroid formation where multiple oscillatory structures embedded in a microfluidic device, such as the one used here, can each form a spheroid in stop-flow condition. Thus, in certain embodiments, the injecting of the mixture at 402 may include the introduction of MC into the mixture. In certain embodiments, the mixture injected thus includes methylcellulose. In alternate embodiments, MC may be introduced separately from the injected mixture. Methylcellulose may be used for the continuous flow condition. The methylcellulose may be added to the cell-biomaterial mixture before the injection into the microfluidic system. The methylcellulose may be introduced in the mixture prior to the injection of the mixture into th channel 18. Alternatively, the methylcellulose may be introduced into the mixture after the mixture has been injected into the channel 18.

Since parallel spheroid formation in stop-flow conditions requires an incubation time and thus a temperature-controlling setup to ensure cell viability, the methodology was adapted to accelerate spheroid formation in continuous flow. Under continuous flow conditions, the physical arrangement of cells under acoustic force is a matter of seconds, and therefore the rapidity with which the cells are adhered together is very important, as it dictates the coagulation time. To promote cell-cell or cell-material adhesion under continuous flow with minimum time, various biomaterials with different mechanisms of adhesion were tested, namely True gel 3D, gelMA, alginate, and poly-L-lysine solution along with Type I collagen.

True gel 3D, a customizable hydrogel that contains slo-Dextran and PEG was used with diluted concentrations to maintain them as a fluid. Small clusters of cells could be formed in the channel using True gel 3D. Cells suspended in a gelMA (methacrylated gelatin)+LAP photo-initiator solution, could be acoustically trapped and form aggregates when exposed to UV. However, the crosslinked aggregates attached to the side-walls and, in some cases, were not easily retrievable. The retrieval of formed aggregates was also challenging with alginate and poly-L-lysine solution (PLL). Coating the channels with Parylene-C at least partially alleviated this issue attesting that both gelMA and Alginate+PLL still present good candidates for promoting the spheroid formation. Finally, type I collagen showed the potential to form rapid adhesion between cells simultaneous to cell trapping. The formed spheroids were easily retrievable, once the optimization of all parameters was achieved. The time and initial concentration of the collagen may be the parameters that were optimized to provide a time window of operation. The concentration of methylcellulose may also be optimized. Given the superior performance of collagen both for acoustic assembly of spheroids and as a natural ECM, as well as considering that it does not require additional steps such as washing between alginate and PLL or UV crosslinking for GelMA, collagen I was selected as the optimal matrix for further investigation in spheroid formation.

In some embodiments, the biomaterial may require cross-linking. Such a cross-linking may be achieved via UV, physical cross-linking, or chemical cross-linking.

Thus, the method 400, at the step 402 of injecting the mixture includes injecting the mixture containing a bioink. In the present embodiment, the bioink selected was Type I collagen. Any suitable bioink may alternatively be used. The bioink may include, for instance, PureCol™ collagen, CELLINK™, and GelMA™. The bioink may include a solution of Type I collagen and Type III collagen. In some cases, the solution includes about 97% by volume of Type I collagen with a remainder of Type III collagen. The collagen may be part of an extracellular matrix. Collagen I is the most abundant and foundational component of ECM. The triple helix proteins of collagen interact laterally and end to end to structure fibrils that support cells while its plethora of cell-binding ligands mediates the cell-collagen adhesion. Moreover, the collagen inherent ability to recreate the complexities of cell-ECM communication may allow cells to interact with dynamic mechanical forces and chemical cues. This active cell-matrix interaction can regulate both the collagen properties through mechanisms such as metalloproteinases (MMPs) degradation, as well as cell phenotypes such as proliferation, polarization, and particularly, metastasis and invasiveness in cancer cells and stemness and differentiation in stem cells.

Some of the bioinks listed above, such as GelMA, may require a control of the temperature within the channel for acoustic microstreams creation. UV may be used for the crosslinking when using GelMA. For some other bioinks, such as CELLINK, strong microstreams may be formed when using high voltages, such as about 90 Vpp or more.

The injecting of the mixture at 402 may include injecting the mixture including the cells embedded in a collagen mixture. The method 400 includes preparing the collagen mixture by obtaining a solution of acid solubilized collagen, and neutralizing the solution with sodium hydroxide to obtain the collagen mixture. More specifically, the cell collagen mixture was prepared through neutralization of acid solubilized collagen and 10× media by sodium hydroxide, followed by the addition of cells in the collagen solution. In this condition, collagen fibrils self-assemble at the cell surfaces and form networks with single or multiple cells trapped in the collagen network.

When this cell-collagen solution is introduced into the microfluidic channel and reaches the acoustic region, the microstreams act as a spheroid assembly line where they trap and compress cells in the eye of the vortices while the collagen fibrils induce rapid adhesion between cells as they make physical contact. Upon reaching the spheroid size of interest, which can be monitored and controlled by trapping duration and flow rate, the acoustic force can be switched off. The assembly process may not be sensitive to cell concentration and the spheroids could be assembled with a wide cell population range of 0.3 to 2 million cells per millilitre. The critical factor in the process, however, is the gelation time. The neutralized collagen molecules self-organize into a network at room temperature and the kinetics of this process directly influences the fluidity of collagen solution and its adhesiveness.

### Generating Vortices

In the embodiment shown, the step of generating the vortices at 404 includes generating the vortices with acoustic waves (acoustic vibrations). Thus, the method at 404 may include generating the vortices by generating acoustic vibrations in the mixture to form acoustic microstreams in the mixture. In the present embodiment, the generating of the vortices using the acoustic vibrations includes inducing vibrations of the sharp edges 21C extending within the channel 18 with the piezo transducer 23. More specifically, in the present embodiment, to create strong vortices, oscillatory bubbles and sharp edges are combined. The acoustic mixer 10 described above with reference to FIGS. 1-3 may be used for this purpose. The combination of the two features (oscillatory bubbles and sharp edges) may create a phenomenon that is stronger than the mere superposition of their effect. The bubbles may diminish the viscous resistance against the sharp edges oscillation while the movement of sharp edges also contributes to the volumetric pulsation of the bubble. The combinatory platform showed considerably stronger microstreams compared to each feature separately, which then allows trapping of various sizes of particles in the vortex at higher flow rates.

In other embodiments, the vortices or microstreams may be generated using one or more of bubbles, sharp edges, any vibratory device, acoustic wave traveling through the channel, and so on. Any suitable combinations of the above may be used to generate the microstreams.

In the present embodiment, the suspended particles such as cells within the collagen or any other suitable biomaterial, when encountering these acoustic microstreams, experience hydrodynamic forces that alter their straight path line. When the microstreams become strong enough, these hydrodynamic forces can overcome the momentum inertia of particles and drag force of background flow to trap cells in the vortex eye.

The intensity of the microstreams may be tuned by controlling the driving voltage during the experiment. That is, the intensity may be varied by varying the voltage supplied to the piezo transducer. The higher voltage may lead to stronger microstreams whereas increasing the flow rate through the channel 18 may tend to suppress the microstreams domains. This results in a trade-off between these two factors in which increasing the input voltage, expands the microstreams domains to cover the whole channel and trap almost all cells. In contrast, increasing the flow rate may lead to the suppression of the microstreams domains, which allows the cells far from the oscillatory complex to escape the acoustic trap. In the present embodiment, the voltage supplied to the piezo transducer may range from 1 to 200 Vpp, preferably from 1-40 Vpp, preferably from 10 to 15 Vpp, preferably about 10 Vpp. It is desired to maintain the voltage as low as possible to limit heat generation, which may alter the cells.

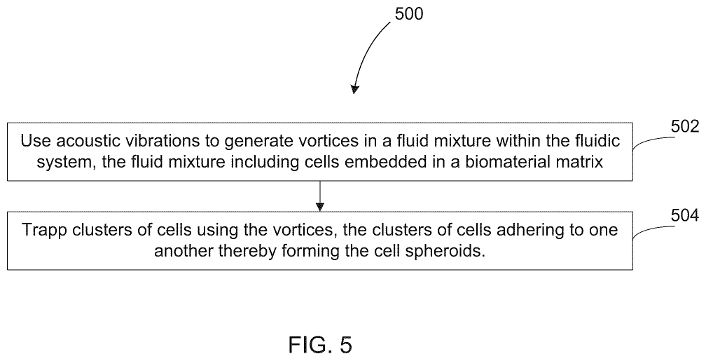

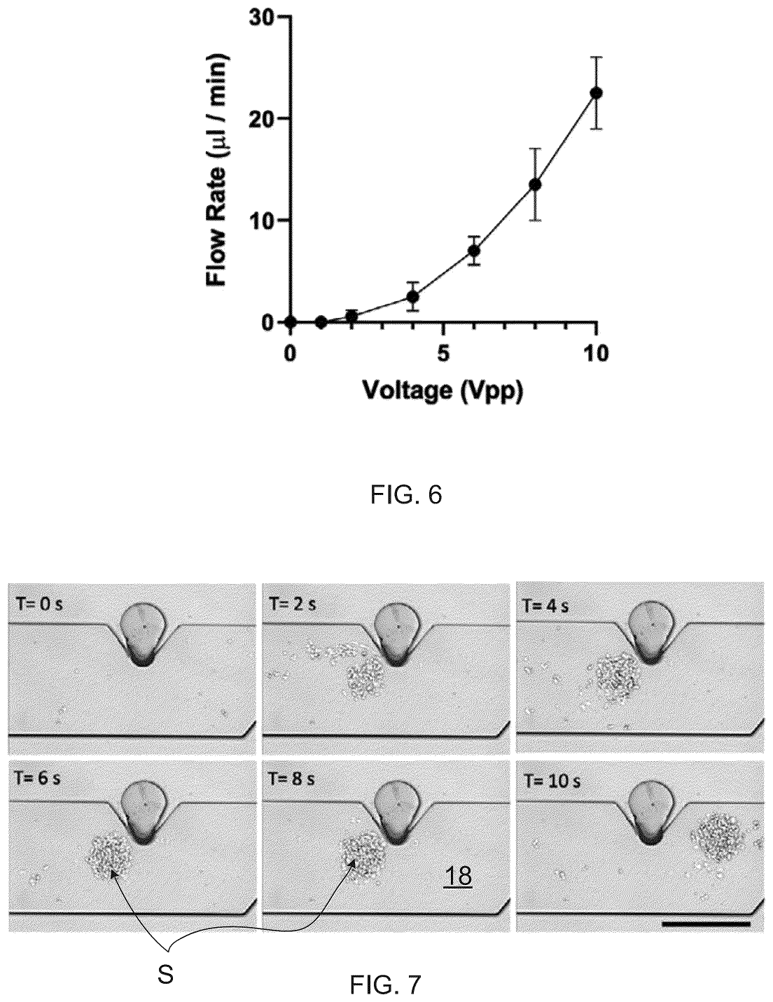

However, increasing the voltage may allow to increate the flowrate through the channel 18, thus increasing an output of spheroids. FIG. 5, depicts the relationship between the voltage and its maximum corresponding flow rate, where cells remain trapped in the vortex. As it can be seen, the intercept of the curve starts from 1 Vpp. This is due to the fact that the microstream intensity below the intercept value is not strong enough to cover the whole channel width and to overcome the momentum inertia of the cells. Thus, the speed of the acoustic spheroid assembly can be controlled by regulating these two factors (voltage and flow rate), considering that applying higher voltages allows higher flow rates and therefore faster trapping, aggregating, and releasing of the spheroids.

Another factor to be considered before initiating the spheroid formation is the effect of acoustic microstreams on the viability of the cells. The main impact of acoustic streams on cells is the shear stress in the acoustic domain. High shear stress can lead to membrane rupture and cell lysis. Since, the magnitude of the shear stress is proportional to acoustic streaming velocity, it can be controlled by the input voltage provided to the piezo transducer. In some embodiments, the voltage may be 10 Vpp at a flow rate of 5 μL min−1.

### Trapping the Cells

In some embodiments, the trapping of the cells at 406 may include stopping the flow through the channel 18 and generating the vortices until the spheroids are formed. This may include stopping the acoustic vibrations once the cell spheroids reach a desired size thereby allowing the cell spheroids to move toward an outlet of the microfluidic channel.

In some other embodiments, the mixture may flow continuously through the channel and the spheroids may be formed with the generated vortices. Thus, the trapping of the cells into the clusters of cells may include continuously flowing the mixture within the channel 18 while the cell spheroids are being formed. In the continuous flow condition, when the vibrations start, the microstreams and their hydrodynamic forces may be strong enough to overcome the momentum of background flow (i.e., the flow from input to output) and trap the cells to form spheroids. When the spheroids or cluster of the cells reach the desired size, the vibrations may be stopped and the cells are free to follow the path of the background flow to move toward the outlet 18B of the channel 18.

The operation window for the acoustic spheroid assembly is the time period that collagen solution remains liquid while the collagen fibril networks are formed around the cells. This may provide the necessary adhesiveness to allow cells to remain attached to each other after the removal of the acoustic force. Exceeding this time window, the collagen solution may be converted into a two-phase solution, consisting of a gelly fiber network with cells, and a liquid portion depleted of collagen fibrils. In such cases, the collagen loses its adhesive role in that the fully gelled network phase is too rigid to infuse in the channel or to be restructured by acoustic shear stress into a spheroid, whereas the liquid phase lacks collagen fibrils to prompt the cell to cell adhesion. This gelation window is highly dependent on the initial concentration of collagen. Understandably, collagen as a natural biopolymer shows batch to batch variation, and therefore, an ever-accurate collagen concentration for the acoustic assembly cannot readily be defined. At lower concentrations, the gel transition is slower, giving a longer window of operation for acoustic spheroid assembly. At concentrations below 0.42 mg mL−1, the collagen solution could not act as an adhesive, and a longer incubation time was required to form cell clusters. However, maintaining cells at room temperature for a long period of time can be detrimental to cell viability. Increasing the incubation temperature to 37° C. showed to considerably accelerate the gelation time, and thereby favors cell to cell attachment. Nevertheless, one should also consider that gelation at physiological temperature might cause changes in fibrils bundling and the collagen structure.

Furthermore, the media used for diluting collagen was also a decisive factor for both the time and quality of the gelation. The addition of 10% of fetal bovine serum (FBS), as used in complete media, accelerated the gelation time significantly. Moreover, this addition generated lumps of the cell-collagen network, causing clogging in the inlet of the microfluidic device. To address this issue, methylcellulose was used to avoid lumps. Interestingly, it also prolonged the gelation time, thus providing more time for spheroid formation. For finding the optimized concentration of MC, it should be noted that higher concentration leads to the higher viscosity of the cell-collagen solution, which limits the acoustic microstreaming intensity and domain. The methylcellulose concentration of 0.4% w/v was observed in the experiments to be optimal for increasing the operation window while keeping the acoustic microstreams domain strong enough to cover the channel width at 10 Vpp.

Referring now to FIG. 5, another method for forming cell spheroids in a fluidic system is shown at 500. The method 500 includes using acoustic vibrations to generate vortices in a fluid mixture within the fluidic system, the fluid mixture including cells embedded in a biomaterial matrix at 502; and trapping clusters of cells using the vortices, the clusters of cells adhering to one another thereby forming the cell spheroids at 504.

The method 500 may further include forming acoustic microstreams in the mixture using the vortices. The method 500 may include using a piezo transducer to induce vibrations of sharp edges within a channel containing the fluid mixture. The method 500 may include continuously flowing the fluid mixture through the channel while the cell spheroids are being formed. The method 500 may include injecting a methylcellulose into the fluid mixture.

Referring to FIG. 7, the formation of spheroids in continuous flow condition is illustrated where the cell trapping and reshaping to spheroids can be accomplished as fast as 10 s. In this example, the cells used are either MDA-MB-231 or MCF-7. Immediately after acoustic assembly, the collagen embedded in cell aggregate produced the robust cell-matrix adhesion to protect the unity of the aggregate during retrieval from the microfluidic channel to the Petri dish.

### Retrieving the Spheroids

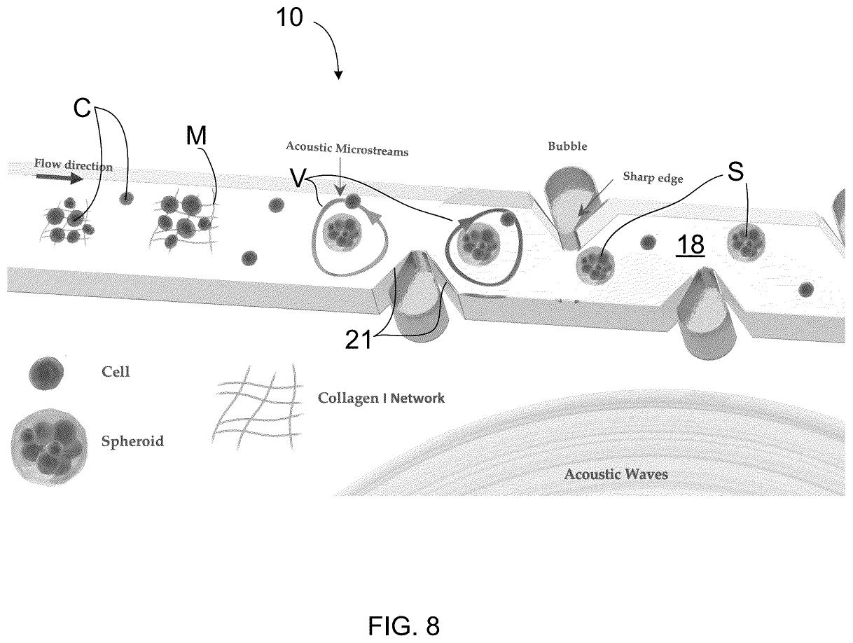

Referring to FIG. 8, the spheroid formation and retrieval process is illustrated. As shown, the mixture including the cells C and the biomaterial M flows towards the protrusions 21 that define the sharp edges. At which point, the cells C become trapped in the vortices C. The resultant cell spheroids S continue to flow toward the outlet of the channel 18 for subsequent retrieval.

The retrieval of acoustically formed spheroids may be straightforward without requiring any pipetting or any additional steps. Upon removal of acoustic streaming, the collagen network is sturdy enough to hold the cells together, and the formed spheroids follow the flow direction to the outlet of the channel 18 and are collected in a Petri dish for further manipulations. The Petri dish may be coated with PolyHema to avoid spheroids' attachment to the substrate and was covered with parafilm to keep sterility during the acoustic assembly. The spheroids are then re-suspended in fresh media and incubated for further compaction and growth. A common challenge faced during the culture of spheroids in a gel-free medium, is the amalgamation of spheroids together and the formation of big clumps. In the present embodiment, spheroids are more susceptible to clumping, especially in the first 2 days, due to the abundance of collagen in the spheroids. To prevent the undesired clumping, the retrieved spheroids, the Petri dish was filled with 1% MC in complete growth media. This may help to minimize the movement of spheroids due to the high viscosity of the milieu. The spheroids incubated in regular media fused together and spheroids incubated in media with MC remained as individuals. However, one should note that the high fusion capacity of spheroids could present an interesting option for tissue engineering and for their 3D printing of cells where the creation of more complex biomimetic tissue is required.

In some embodiments, the retrieving of the cell spheroids from the channel at 408 includes flowing the spheroids out of the channel 18 by injecting a fluid into the channel 18 to push the spheroids towards the outlet of the channel 18. The fluid may be, for instance, phosphate-buffered saline or Dulbecco's modified Eagle medium.

The disclosed method may allow the bioprinting, mixing many types of cells together, may not rely on cell type for the creation of spheroids, non-cell particles may be included in the spheroids, for instance, each of the two inlets of the acoustic mixer 10 may receive a respective one of two cell types, or one cell type and another non-cell particle.

### Spheroid Culture and Analysis of Cell Survival/Functionality

The size development and morphological evolution of spheroids formed by acoustic assembly with MDA-MB-231 cell line and MCF-7 cell was analyzed. During the first hour of cultivation, individual cell boundaries are discernible. After a few hours in culture and until a day of incubation, the aggregates' size shrinks, and their boundaries become less distinguishable. The smooth surface of the cell cluster indicated the formation of monolithic spheroids.

According to the initial cell concentration in aggregates, the expected diameter of spheroids was around 200 micrometers immediately after the assembly. However, a variation in their size was observed over the cultivation period. The mean diameter of spheroids over time showed a similar pattern of decrease in the size of the spheroids of both cell types during the first day. The decrease in the diameter is attributed to the reconfiguration of cells in the spheroids in combination with the compaction phase which in the cells create tighter junctions by secretion of integrins and/or cadherins. The compaction phase is followed by the proliferation of cells that leads to gradual growth in the diameter of the spheroids with slightly higher growth for MDA-MB-231 spheroids compared to MCF-7 spheroids.

The spheroids of both cell-line show almost similar round morphology over 5 days of culture. MDA-MB-231 cells generally compacted faster and showed cells at peripheral of spheroids after 4 days. This tendency of MDA-MB-231 cells to migrate out of the spheroids can be attributed to their higher invasiveness. While the well-defined spheroids of MCF-7 cells is commonly observed in all methods, the compact spherical morphology of acoustically assembled MDA-MB-231 spheroids was interesting, as this cell type usually remains in loose form due to the weak cell-cell adhesion in other methods of spheroid formation. To further emphasize the quality on cell-cell adhesion with our methodology, the shaking plate was used to form spheroids with MDA-MB-231 and confirm the loose aggregation.

The difference in the quality of cell aggregates between MDA-MB-231 and MCF-7 cells stems from the compaction mechanism of these spheroids. MCF-7 cells secrete and accumulate E-cadherin on their surface to promote compact junctions through homophylic cadherin-cadherin bindings, in contrast, these binding molecules have no participation in the compaction of MDA-MB-231 spheroids. E-cadherin molecules were visible at cell junctions in the MCF-7 spheroids, but are absent in MDA-MB-231 spheroid even though they also form compact spheroids in acoustic assembly. Since in the acoustic spheroid formation cells are surrounded by collagen I, it is believed that the presence and interaction of collagen I with integrin b proteins has played an essential role in the compaction of MDA-MB-231. From this result, one can conclude that the acoustic assembly is compatible with both cell lines despite their difference in the compaction mechanism.

The MCF-7 and MDA-MB-231 cell viability in spheroids was determined by live/dead assay kit. The result showed that the cell viability was maintained even after 7 days. Some dead cells were distributed through the spheroids, but they did not seem to indicate any necrotic core. This can be due to the relatively small size of spheroids as well as the active assembly of the cells, which was shown to prevent the formation of necrotic cores. Interestingly, image analysis of cell viability over 7 days indicated that the cell viability in spheroids formed immediately after acoustic exposure is higher than the cell viability of individual cells at 10 Vpp. This can be attributed to the high tensile strength of collagen network that can act as a shield and protect the cell membrane from shear stress induced by the microstreams. Collagen fibrils surrounding the suspended cells can also sup-port them against cell death due to non-adherence, as reported by Shin et al. with polymer nanofibers.

### Features of Spheroid Formation in Acoustic Microstreams

The demonstration of the acoustic formation of coherent spheroids relying on homophilic cadherin-cadherin interactions through the use of collagen I, may open the possibility for the formation of multicellular, heterotypic spheroids or cell-particle spheroids, regardless of their biological or inorganic nature. The experiment allowed to observe the multicellular spheroids of MCF-7 cells which normally form homotypic cell-cell adhesion mediated by E-cadherin and MDA-MB-231, which do not express E-cadherin and their aggregation relies on integrin b-collagen I binding. This purely physical assembly strategy may allow to juxtapose and co-culture consortia of multi cell-lines or multi-species cells for creating synthetic crosstalk between cells. Moreover, one of the challenges in studying multicellular spheroids is the variant extracellular environment which can be mitigated by the con-trolled incorporation of collagen.

Cell-particle composite spheroids have been used in numerous applications from guiding the spheroids in a magnetic field by incorporating magnetic particles to sensing or regulating mechanical properties, increasing cell viability, and inducing differentiation in stem cells. Centrifugation or gravity-based methods to incorporate particles may be used. However, in the likely case of disparity between the density of cells and particles, it leads to sedimentation and therefore, uneven distribution of particles and cells. Additional steps and equipment such as random positioning machine are required to improve the quality of the composite spheroids. In the present disclosure, the acoustic assembly platform is used to alleviate the uneven distribution challenge by homogenizing the multi-cells or cell particle mixtures simultaneously to the spheroid formation. The homogeneous and densely packed spheroid of 5 μm diameter polystyrene particles (green) with an approximate total of 3666.7±590.2 and MDA-MB-231 cells was overserved. Moreover, since the interfaces between cells and microparticles are supported by collagen fibrils in acoustic assembly, it may obviate the need to conjugate RGD peptides or collagen fibrils to particles' surface to ensure their attachment into the spheroid. The forced and random positioning of cells in multicellular spheroids both with or without particles can be specifically helpful for studying the morphological change and migration behavior of cell types over time, due to differences in cadherin and integrin expression levels in a spheroid.

Two prerequisites for the use of spheroid as building blocks in tissue engineering are i) robust ECM to ensure stability and integrity of spheroids during the process and ii) the adhesiveness of spheroids to initiate fusion. However, during spheroid development, these two parameters progress inversely: as the spheroids mature and ECM deposition surges, adhesiveness among spheroids decreases. Another factor adding to the complexity of using spheroids as building blocks, is the viability of cells since during the time needed to form mechanically stable spheroids, the core cells can be deprived of oxygen and nutrients. The ability of the disclosed acoustic mixer 10 to incorporate collagen in a well-controlled manner can help to simultaneously promote both mechanical stability and adhesiveness of spheroids. It was observed that the spheroids retained their unity under shear stress immediately after formation. Moreover, the addition of collagen I as the adhesion-promoting factor can be used to study and control the kinetics of spheroid fusion. To demonstrate this capability, the spheroids of MDA-MB-231 stained by Deep Red Cytopainter and MCF-7 stained with Green CellTracker CMFDA were cultured together in a 35 mm Petri dish within an hour of their formation. After 24 h in the culture, both cell types retain their integrity but the spheroids were starting to merge. The rapid production of high cell-density spheroids and their ability to immediately be used as building blocks can address some of the challenges of slow-growth spheroid formation such as deprivation of oxygen and nutrients to the cells over time.

### CONCLUSION

Although the present disclosure focuses on generating spheroids from cancer cells (tumoroids), the present technology may also be used for mixing cells, cells and particles, and making cell-particle spheroids composites and so on.

A rapid and matrix-supported spheroid formation method using an acoustically-driven microfluidic platform is described herein. This method may allow cells to aggregate in the eye of the vortex V which can be used independently as a cell trapping/enrichment system or as a spheroid formation device. By adding collagen as a bioadhesive, the acoustic platform may shape and support cells into a 3D spheroid in seconds and recapitulate the native growth environment. The acoustic mixer 10 may allow for physical assembly and homogenous agglomeration of multi-types of cells or even particles in the vicinity of each other for studying cell behaviors such as migration, crosstalk, or changes in the morphology.

The closely packed acoustic assembly may hold the potential to overcome some of the spheroids formation challenges especially for bottom-up tissue engineering such as low density, lack of cell-matrix, and cell-cell communication after formation, or reduced fusion ability after maturation. Moreover, this technique may open the venue of regulating both the mechanical and chemical characteristics of the growth microenvironment by additional steps such as crosslinking or encapsulating chemical cues in the collagen. The platform can also be used for shear stress studies on both spheroids and cells by applying controllable acoustic forces. The stability of rapidly formed spheroids combined with their high fusion tendency along with the ability of our device for making composite spheroid with functional particles such as magnetic particles that can be used for the directed fusion of spheroids, offer the possibility of creating complex tissue structures as models to investigate the underlying mechanisms of various diseases and develop the treatment modalities.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

1. A method for forming cell spheroids in a fluidic system, comprising:
injecting a mixture including cells embedded in a biomaterial matrix into a channel;
generating vortices in the mixture flowing within the channel;
trapping the cells using the vortices to form clusters of cells until the cells of the clusters of cells adhere to one another via the biomaterial matrix thereby forming the cell spheroids; and
retrieving the cell spheroids from the channel.

2. The method of claim 1, wherein the generating of the vortices includes generating acoustic vibrations in the mixture to form acoustic microstreams in the mixture.

3. The method of claim 2, wherein the generating of the vortices using the acoustic vibrations includes inducing vibrations of sharp edges extending within the channel with a piezo transducer.

4. The method of claim 1, wherein the trapping of the cells into the clusters of cells includes continuously flowing the mixture within the channel while the cell spheroids are being formed.

5. The method of claim 4, wherein the generating of the vortices includes generating the vortices with acoustic vibrations.

6. The method of claim 5, further comprising stopping the acoustic vibrations once the cell spheroids reach a desired size, thereby allowing the cell spheroids to move toward an outlet of the channel.

7. The method of claim 1, wherein the injecting of the mixture includes injecting the mixture including the cells embedded in a collagen mixture.

8. The method of claim 7, wherein the collagen mixture includes Type I collagen.

9. The method of claim 7, further comprising preparing the collagen mixture by:
obtaining a solution of acid solubilized collagen; and
neutralizing the solution with sodium hydroxide to obtain the collagen mixture.

10. The method of claim 1, wherein the injecting of the mixture includes injecting the mixture having a cell concentration of from 0.3 to 2 million cells per millilitre.

11. The method of claim 1, wherein the retrieving the cell spheroids includes flowing the cell spheroids out of the channel by injecting a fluid into the channel to push the cell spheroids towards an outlet of the channel.

12. The method of claim 11, wherein the injecting of the fluid includes injecting phosphate-buffered saline or Dulbecco's modified Eagle medium.

13. The method of claim 1, further comprising introducing methylcellulose into the mixture.

14. The method of claim 13, wherein the methylcellulose is introduced into the mixture prior to the injection of the mixture into the channel.

15. The method of claim 13, wherein the methylcellulose is introduced into the mixture after the mixture has been injected into the channel.

16. A method for forming cell spheroids in a fluidic system, comprising:
using acoustic vibrations to generate vortices in a fluid mixture within the fluidic system, the fluid mixture including cells embedded in a biomaterial matrix; and
trapping clusters of cells using the vortices, the clusters of cells adhering to one another thereby forming the cell spheroids.

17. The method of claim 16, further comprising forming acoustic microstreams in the mixture using the vortices.

18. The method of claim 17, further comprising using a piezo transducer to induce vibrations of sharp edges within a channel containing the fluid mixture.

19. The method of claim 18, further comprising continuously flowing the fluid mixture through the channel while the cell spheroids are being formed.

20. The method of claim 16, further comprising injecting a methylcellulose into the fluid mixture.
